(19) United States
(12) Patent Application Publication
(10) Pub. No.: US 2020/0121391 A1
(43) Pub. Date: Apr. 23, 2020

(54) SYSTEM AND METHOD FOR PLANNING AND EXECUTING AUTOLOGOUS BONE GRAFTING PROCEDURES

(71) Applicant: THINK SURGICAL, INC., Fremont, CA (US)

(72) Inventors: Micah Forstein, Fremont, CA (US); Kyle Kuznik, Fremont, CA (US); Hosna Akhlaghpour, Fremont, CA (US); Jack Ulstad, Fremont, CA (US); Amit Sandhu, Fremont, CA (US); Mohammad Najafi, Fremont, CA (US)

(21) Appl. No.: 16/500,638

(22) PCT Filed: Mar. 27, 2018
(86) PCT No.: PCT/US2018/024554

§ 371 Date: Oct. 3, 2019

Related U.S. Application Data
(60) Provisional application No. 62/481,189, filed on Apr. 4, 2017.

(51) Int. Cl. A61B 34/10; A61B 34/30

(52) U.S. Cl. CPC A61B 34/10; A61B 2090/3762; A61B 34/30

(57) ABSTRACT

A method for planning a bone grafting procedure includes the collection of imaging scan data of the target region and of a harvest region as a source for a bone graft complementary to the target region. Virtual bone model and harvest regions are generated. A computer processor determines at least one parameter for a bone graft model complementary to the target region. A location at the harvest region for the bone graft is identified based on the bone graft model. The location of the harvest region is registered to a first computer-assist device and the bone graft is harvested. The target region is registered to the first computer-assist device or another device. The cutting characteristics for the target region are communicated to the first computer-assist device or the other device to receive the bone graft. A surgical system for performing the computerized method is also provided.

### RELATED APPLICATIONS

This application claims priority benefit of U.S. Provisional Application Ser. No. 62/481,189 filed 4 Apr. 2017, the contents of which are hereby incorporated by reference.

### FIELD OF THE INVENTION

The invention relates generally to the field of computer-assisted orthopedic surgery, and more specifically to a new and useful system and method for planning and executing bone grafting procedures.

### BACKGROUND OF THE INVENTION

Bone grafting is a surgical procedure in which replacement bone is placed into spaces around a broken bone or in between holes or defects in the bone. Bone grafting may be required for any number of reasons, for example, bone fractures with bone loss, repair of bone that has not properly healed, maxillofacial reconstruction, and treatment of joints to prevent movement (fusion). Bone graft material may include autogenous bone (autograft), allograft, xenograft, or synthetic bone graft substitute. In many orthopedic surgical procedures, the use of autografts are preferred over other types of grafts since autografts include osteogenic cells, osteoinductive growth factors, and an osteoconductive scaffolds, all essential for new bone growth. In addition to the osteogenesis advantages, autografts do not carry the risk of disease transmissions and immunological rejections. Autographs however, have a high percentage of morbidities at the harvesting site and there is limited shape availability.

Morbidities associated with the autografts may be the results of arterial injury, herniation, nerve injury, and hematoma that occur during the harvesting. Also, there is a lack in precision in identifying an optimum harvesting location, and the current design of many common cutting tools make it difficult to create accurate, non-planar, and/or detailed shapes for a graft.

Currently, conventional methods for designing and cutting autografts are simply sections of bone not well suited or specifically designed to precisely fit in the targeted bone region (i.e., the region of bone requiring treatment, surgery or replacement), nor can the autograft and target site be prepared with enough accuracy to optimize bony contact and autograft stability. As a result, the fusion of the harvest bone with the target bone is sub-optimal. A surgeon may instead use an allograft that is shaped and sized to customize the allograft for the subject's target bone anatomy; however significant shaping and sizing of an autograft is not possible due to the nature of the autograft and lack of precise methods. Even if extensive shaping and sizing were possible, a surgeon's ability to manually shape and size the allograft to the desired dimensions is severely limited based in part on the limited functionality of the conventional tools available in the operating room. The limited shape of the autograft, typically harvested with tools that create planar cuts (e.g., osteotomes, surgical saws), also makes it difficult for the surgeon to identify the proper position for the graft in the target region, leaving some uncertainty in the final outcome of the subject.

Various techniques have been developed to help a surgeon plan and execute cartilage replacement procedures. One system and method for creating unique patterns for cartilage plugs is described in U.S. Patent Publication 2016/0038291 assigned to the assignee of the present application. With reference to FIG. 1, a method 100 utilizes a computer assisted surgical system and a subject specific surgical plan to create a precise cavity for a cartilage replacement implant. The process 100 includes the steps of: receiving scan data of a subject's bone and creating a virtual three-dimensional model of the subject's bone and cartilage including the cartilage defect (Block S110); creating a custom shape around the cartilage defect to be removed based on the virtual model (Block S120); creating or shaping a pre-made cartilage replacement implant to match the custom shape (Block S130); registering the location of the actual bone during the surgery such that the precise position and orientation of the bone is known by the robot (Block S140); robotically milling the custom shape into the bone in the location predetermined in the virtual model (Block S150); and finally placing the graft into the bone (Block S160). Although this system introduces an effective technique for creating a custom cavity into the bone to receive a cartilage implant, the method requires a pre-made cartilage implant that is formed and shaped prior to the surgery using conventional machining techniques. Although the method suggests the use of autologous materials to create the pre-made cartilage implant, two separate procedures may be required including a first procedure to harvest the autologous material, which is subsequently processed to the desired shape, and a second procedure to implant the graft at the target region.

While allografts are traditionally formed in a finite number of standard sizes from sterilized cadaver bone, these inserts are traditionally wedged into position with gaps being filled with bone chips or other materials stimulative of osteoclast infiltration into the allograft. Allograft machining is labor intensive and prone to poor fit.

Thus, there is a need in the art for a system and method to plan and execute bone grafting procedures with a particular emphasis on the machining, mating, and assembling of bone fragments regardless of whether the bone is autologous or cadaverous in origin. There is a further need to more effectively identify and/or process grafting material, specifically the identification and preparation of healthy autologous bone for harvest and to reduce the chance of surrounding bone morbidity. There is an even further need to improve the attachment and integration of two or more bone fragments to form a mechanically effective, strong unit.

### SUMMARY OF THE INVENTION

A method for planning a bone grafting procedure for a subject bone having a target region. The method includes the collection of imaging scan data of the target region and of a harvest region as a source for a bone graft complementary to the target region. A virtual bone model of the bone is generated, as is a virtual model of the harvest region. A computer processor determines at least one of a size, a material type, a geometry, and a position for a bone graft model complementary to the target region. A location at the harvest region for the bone graft is identified based on the bone graft model. The location of the harvest region is registered to a first computer-assist device to harvest the bone graft. The bone graft is harvested from the harvest region with the first computer-assist device. The target region is registered to the first computer-assist device or a second computer-assist device. The cutting characteristics for the target region are communicated to the first computer-assist device or a second computer-assist device to form the target region to receive the bone graft.

A surgical system for performing the computerized method is also provided.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 is a flowchart depicting a prior-art method for creating a custom shaped cavity for a cartilage replacement implant;

FIG. 2 is a flowchart depicting a method for planning and executing a custom bone grafting procedure in accordance with embodiments of the invention;

FIG. 3 is a flowchart depicting a method for planning and executing a custom bone graft having joint features in accordance with embodiments of the invention;

FIG. 4A illustrates a custom bone grafting procedure for replacement of a portion of a mandible in accordance with embodiments of the invention;

FIG. 4B illustrates a custom bone grafting procedure for replacement of a portion of a mandible with a custom bone graft having joint features in accordance with embodiments of the invention;

FIG. 5 illustrates a custom graft having a dovetail joint feature in accordance with embodiments of the invention;

FIG. 6 illustrates a custom graft having a mortise or tenon joint feature in accordance with embodiments of the invention;

FIG. 7 illustrates a custom graft and target region have an inset and an attachment feature in accordance with embodiments of the invention;

FIG. 8A illustrates a custom fixation plate for use with the custom grafting procedure in accordance with embodiments of the invention;

FIG. 8B illustrates an inset milled in a custom graft and a target region to receive a fixation plate in accordance with embodiments of the invention;

FIG. 9 illustrates a custom graft having object features in accordance with embodiments of the invention;

FIG. 10 illustrates a robotic surgical system for executing a bone grafting procedure in accordance with embodiments of the invention; and

FIG. 11 depicts systems and methods for executing a bone grafting procedure wherein a cadaverous graft is prepared at a remote commercial laboratory in accordance with embodiments of the invention.

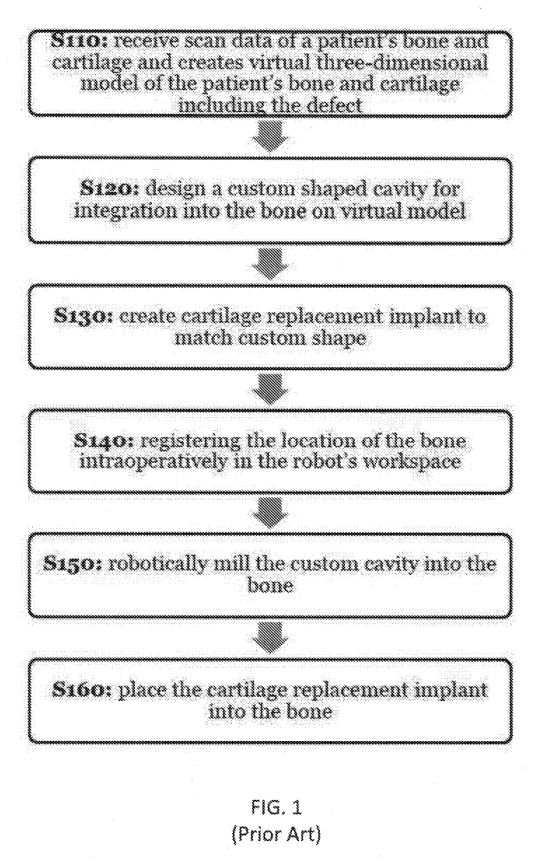

### DESCRIPTION OF THE INVENTION

The present invention has utility as a system and method for planning and executing bone grafting procedures. The system and method is especially advantageous for complex cases requiring the replacement of missing bone, filling gaps in a bone, or bridging of two or more bone fragments together, which is common in procedures such as maxillofacial facial reconstruction, long bone fracture repair, high tibial osteotomies, and vertebra spinal fusion. As reference is made herein to the replacement of a portion of the mandible in maxillofacial surgery specifically, it should be understood that the present invention may be applied to other bones and joints found within the body illustratively including the radius, ulna, humorous, femur, tibia, fibula, the bones of the hand and feet, vertebra, pelvis, skull, sternum, ribs, and each of their associated joints where applicable. It is also contemplated that the system and method described herein is readily applied for use on non-humans. Therefore, as used herein, a ‘subject’ is defined as a human, a non-human primate; or an animal of a horse, a cow, a sheep, a goat, a cat, a dog, a rodent, and a bird; or a cadaver of any of the aforementioned.

The following description of various embodiments of the invention is not intended to limit the invention to these specific embodiments, but rather to enable any person skilled in the art to make and use this invention through exemplary aspects thereof.

All publications, patent applications, patents and other references mentioned herein are incorporated by reference in their entirety. References recited herein are indicative of a level of skill in the art to which the invention pertains.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “registration” refers to the determination of the spatial relationship between two or more objects or coordinate systems such as a computer-assist device, a bone, and/or an image data set of a bone. Illustrative methods of registration known in the art are described in U.S. Pat. Nos. 6,033,415, 8,010,177, 8,036,441, and 8,287,522, and U.S. Pat. App. No. 20160338776.

As used herein, the term “joint” refers to a place at which two things, or separate parts of one thing, are joined or united, to either form a rigid connection therebetween, or joined in such a way as to permit motion without decoupling; juncture.

As used herein, the term “join” or “joining” refers to fitting, interfitting, mating, locking, interlocking, or meshing, all of which are used to generically describe the joining of bone sections or pieces together.

As used herein, the term “joint feature” refers to a feature present on a thing (e.g., a graft or a honey region) intended to form a joint. Various types, sizes, and shapes of these features are described throughout the description.

As used herein, the term “matching joint feature” refers to a second joint feature that fits, adapts, engages, negatively matches, mates, interlocks, or otherwise corresponds to a first joint feature. The matching joint feature may correspond with respect to size, shape, pattern, and nature.

As used herein, the term “target region” refers to an anatomical region in need of treatment, repair, or other surgical intervention.

As used herein, the term “harvesting region” refers to an anatomical region for collecting bone to treat, repair, or replace the target region. An example of the harvesting region may include but not limited to the iliac crest of the pelvic bone, the rib bones, and the fibula, and in some embodiments, cadaverous bone.

While the present invention is illustrated visually hereafter with respect to a mandible as an example of the target bone and a pelvic bone as an example of the harvesting bone for which the present invention is applied, it is appreciated that the present invention is equally applicable to other bones of a human, non-human primate, or other mammals.

Any of a wide variety of different bone grafting materials, particularly autografts, allografts and/or xenograft structures, can be prepared according to the teaching of this invention; however the use of an autograft harvested from a subject in specific embodiments is the preferred grafting material as further described throughout the description.

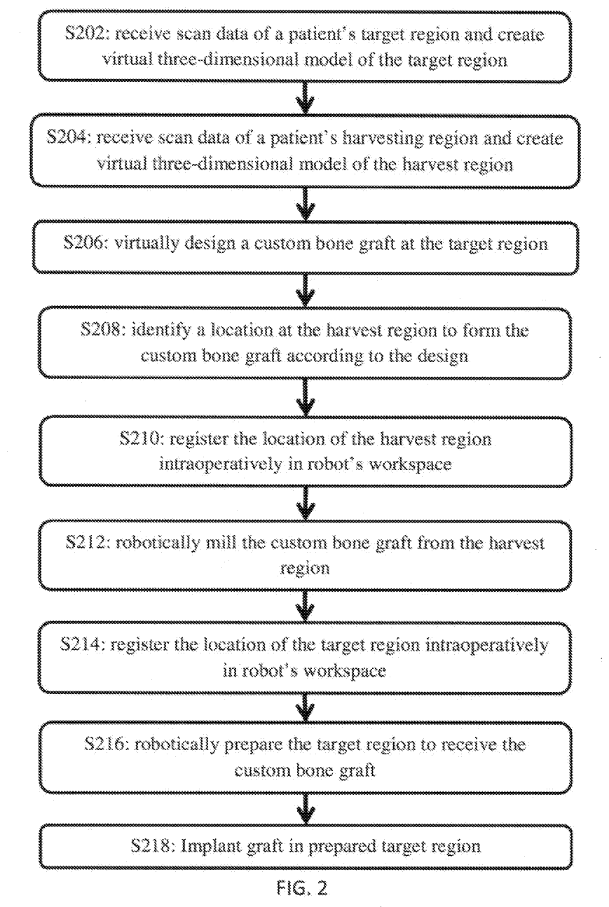

With reference to the figures, FIG. 2 generally depicts a method for planning and executing a bone grafting procedure. Imaging scan data of the subject's target region and harvest region are collected and created into three-dimensional (3-D) virtual models (Blocks S202 and S204). It is appreciated that a harvest region can be a region of subject autologous bone or a cadaver bone. In those instances when cadaver bone is used, a first robot shapes the custom graft and a second surgical robot is used to prepare a target region for receipt of the same. A user may then design a custom bone graft at the target region to treat, repair, or replace the target region (Block S206). The user and/or the computer may manually or automatically identify a location at the harvest region to create the custom bone graft according to the design (Block S208). In specific embodiments, the dimensions and geometry of the custom bone graft is superimposed at the harvest region in a location with good bone quality, accessible by a tool operated by a computer-assist device with minimal tissue damage, and a location with a low probability of surrounding bone morbidity after the bone is harvested. This is especially important for autologous harvest. Intra-operatively, the location of an autologous harvest region is registered to the computer-assist device to accurately harvest the custom bone graft at the identified location and to the specifications of the custom graft geometry (Blocks S210 and S212). Next, the location of the target region is registered to the device (Block S214), and the device accurately prepares the target region to receive the graft (Block S216). Finally, the graft is implanted into the prepared target region (Block S218). In some inventive embodiments, a harvest region is filled with conventional substances that illustratively include bone cement, bone chip, hydroxyapatite, calcium sulfate, tricalcium phosphate, or combinations thereof.

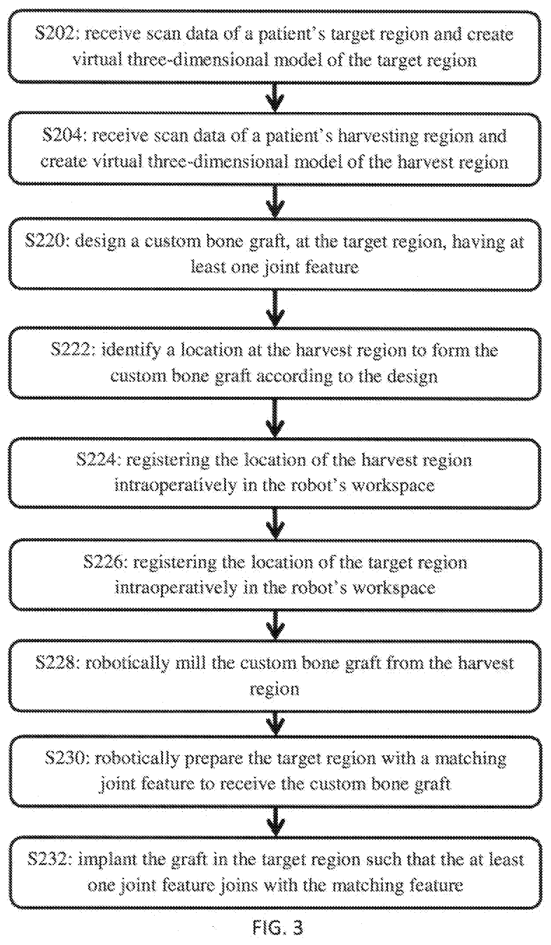

In a specific embodiment, the bone graft and/or target region includes features to improve the connectivity of the graft with the target region and/or include features to aid in aligning the bone graft with the target region. With reference to FIG. 3, a method for the planning and execution of a graft and target region having matching joint features is generally shown. The planning step includes; (a) receiving scan data of a subject's target and harvesting region (Block S202); (b) creating a virtual bone model of the target and harvesting region or an element thereof, which may further contain bone property and soft tissue data (Blocks S204); (c) designing a custom bone graft, at the target region, having at least one joint feature (Block S220); and (d) identifying a location at the harvest region to form the custom bone graft according to the design (Block S222). The execution step includes; (a) registering the location of the target and harvesting regions intraoperatively in the robot's workspace (Block S224 and Block S226); (b) robotically milling the custom bone graft; (Block S228); (c) robotically preparing the target region with at least one matching joint feature to receive the custom bone graft (Block S230); and (d) implanting the graft in the target region such that the at least one joint feature of the graft joins with the matching feature prepared at the target region to form a joint (Blocks S232). Specific embodiments of the system and method are further described in detail below.

Planning

The bone models are obtained (Block S202-S204) by generating a three-dimensional (3-D) bone model from an image data set of the subject's anatomy. The image scan data may be collected with an imaging modality such as computed tomography (CT), dual-energy x-ray absorptiometry (DEXA), magnetic resonance imaging (MRI), X-ray scans, ultrasound, or a combination thereof. The 3-D bone model(s) are readily generated from the image scan data using medical imaging software such as Mimics® (Materialise, Plymouth, Mich.) or other techniques known in the art such as the one described in U.S. Pat. No. 5,951,475.

Scan data of the subject's bone may include any of the structural/anatomical features illustratively including size, shape, thickness, and curvatures. In addition to bony structural features, the scan data may include bone property and soft-tissue data, for example bone alignments, bone kinematics, soft tissue features, placement of nerves and arteries, bone density and bone microarchitecture. Subject-specific features can be identified from analysis of the scan data and segmented images to aid in the design of a custom graft and to identify optimal harvesting regions.

The user is able to view and manipulate the bone model and bone property data in a pre-operative planning software program having a graphical user interface (GUI). The GUI includes widgets and other tools which allow a user to manually, semi-automatically, or automatically design a custom graft and, in some embodiments, identify a location at the harvest region to create the custom graft as further described below.

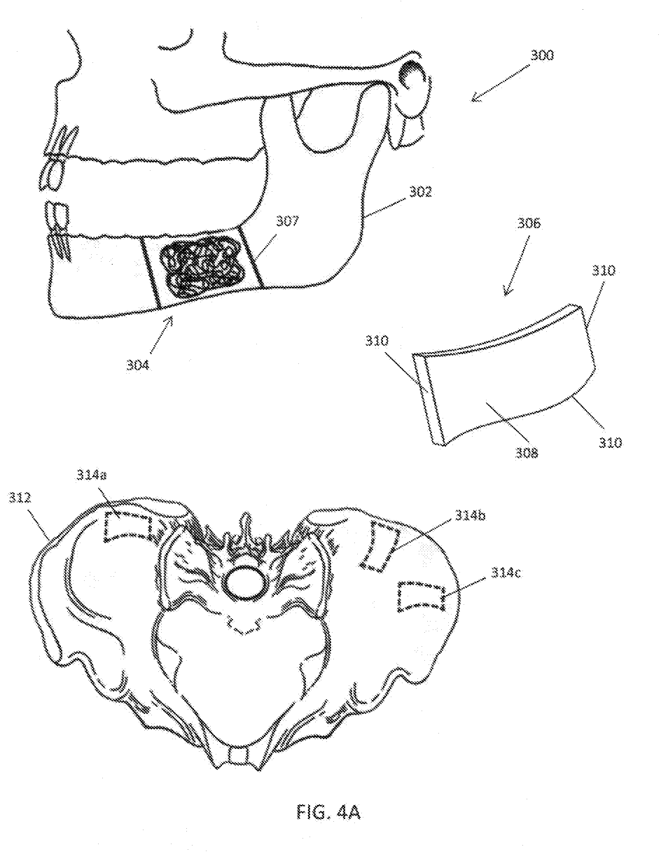

In a specific embodiment, with reference to FIG. 4A, an example of designing a custom graft to replace a portion of the mandible is generally shown at 300. A 3-D model of a target region 302, such as the mandible, is generated from scan data. A user may identify a target region 304 in need of replacement, such as a region of bone having cancerous tumors. With the bone model 302, the user designs the initial custom graft 306 by determining at least one of a size, type, geometry and position for the graft 306. For clarity, the initial custom graft 306 refers to a graft design that replaces a region of bone requiring removal or to restore the function of a subject's joint or bone for the surgical procedure. In a particular inventive embodiment, the GUI includes tools such as splines, lines, and generic shapes with or without modifiable meshed surfaces to design the graft 306. A library of modifiable shapes resembling typical structures of a particular bone (e.g., a generic modifiable shape of a mandible, a generic modifiable shape of a vertebral body) may also be provided in the GUI. In another embodiment, geometric data about the bone model 306 may be extracted to aid in the initial graft design. The topology of the bone model may be extracted to define the natural contours and curvature of the native bone. The contours and curvatures may define at least a portion of the graft shape to match the actual bone topology. The extracted topology data may also originate from the lateral side of the subject and mirrored to create a graft resembling the natural structure of the native bone. In a further embodiment, the GUI may include boundary tools to simply define boundaries 307 on the model 302 to create design the custom graft 306. The boundary tools may include planes, spheres, prisms, or other shapes having control points to adjust their shapes to define the boundaries 307. In one embodiment, the boundary tool may be used to virtually cut out a custom graft 306, where all the voxels inside the boundaries are removed from the surrounding voxels. It should be appreciated that the tools for defining a desired shape (i.e., splines, lines and shapes), the generic models, the geometry extraction tools, and boundary tools may all be available to the user in the GUI. These widgets and tools can also be used for designing the joint features as further described below.

In a particular inventive embodiment, as depicted in FIG. 4A, the initial graft 306 may include a body 308 having one or more sides 310 with no joint features. The sides 310 of the body 308 are non-planar and have a curved profile to better match the native anatomy. A graft having this curved profile is nearly impossible to create using conventional planar shaped tools without any post-processing techniques common to allograft manufacture, which would otherwise increase the operating room time when autograft harvesting.

With the custom graft 306 designed, the user and/or the planning system may identify an optimal location at the harvesting region 312 to harvest the graft 306. A 3-D model of the harvesting region 312, such as the pelvis, is generated from scan data. The 3-D model of the harvesting region 312 may further include bone property data, specifically bone density/quality data. In a specific embodiment, a user may virtually cut out and/or manipulate a model or outline of the custom graft 306 and superimpose or overlap the graft 306 at different locations on the harvest region 312 in order to identify an optimal graft harvesting location (314a, 314b, 314c). The user may take into consideration the quality of the bone to be harvested in order to harvest a graft with good structural integrity. The user may also consider the quality of the bone surrounding the bone to be harvested to reduce bone morbidity following the harvest.

In a specific inventive embodiment, a duplicate of the model/outline of the custom graft 306 is generated. A first model of the graft 306 remains at the target region, while a second model of the graft 306 is manipulated by the user to identify a harvesting location (314a, 314b, 314c). If the user decides to change the design of the custom graft 306 at the target region, then the second model of the graft 306 is automatically updated to reflect that change at a harvesting location. Therefore, the user may quickly update the design of the graft 306 and immediately identify a new location for the updated design (or confirm that the currently identified harvesting location is still adequate).

In a particular inventive embodiment, the GUI includes an indicator to indicate a percentage of overlap between the model/outline of the graft 306 and potential harvesting locations. For example, the indicator may show 95% overlap between the model of the graft and a particular harvesting location. In this situation, the user needs to identify a new harvesting location because only 95% of the shape of the graft 306 can be harvested. Because virtual environments are occasionally difficult to navigate, the indicator ensures the user identifies a harvest location where 100% of the graft 306 is millable, regardless of whether in autologous or cadaver bone.

In a specific embodiment, the planning system may semi-automatically identify an optimal harvest location (314a, 314b, 314c). Based on the geometry of the custom graft 306 designed by the user, the planning system may first identify harvest locations where there is 100% overlap between the graft geometry and the harvesting region 312. The system may then evaluate the bone quality at these harvest locations to identify one or more optimal harvest locations (314a, 314b, 314c). The user may then choose a final optimal harvest location 314b and/or modify the location 314b as desired. Once an optimal harvest location 314b is identified, the position and orientation (POSE) of the graft 306 with respect to the harvesting region 312 (i.e., the pelvis) is saved for use intra-operatively.

Planning—Matching Joints

In another inventive embodiment, with respect to FIG. 4B, the GUI allows the user to design a jointed custom graft 306′. The jointed custom graft 306′ may include a body 308′ with one or more sides 310′ having joint features 326. A jointed custom graft 306′ is particularly advantageous for several reasons. First, the joint feature 326 may help prevent the graft 306′ from separating from the target region 304. Second, an interlocking or overlapping joint, such as with a tongue and groove design as described below, creates a greater surface area for bonding and osseointegration. Third, the jointed custom graft 306′ may minimize the amount of healthy bone margin around the harvesting site to be removed, as opposed to the current methods which is limited to harvesting the bone with planar shaped instruments that may remove more bone than necessary for a given procedure. Finally, having a jointed custom graft 306′ prevents the misplacement of the graft 306′ to the target region 304 during surgery and saves the user's time by providing a reference (i.e., the specific design of the joint features) to quickly align the graft 306′ to the target region 304.

The jointed custom graft 306′ is designed to include one or more joint features 326 that correspond with a matching joint feature 328 prepared at the target region 304. The user may design the jointed custom graft 306′ and identify an optimal harvesting location using the aforementioned GUI and planning system tools. The user may further evaluate the quality of bone in terms of bone density to optimize and/or determine a suitable location for the joint to increase stability and osseointegration. Specific joints features are further described below.

In a specific inventive embodiment, with reference to FIG. 4B, the joint feature 326 may include a plurality of ridges and grooves that mate with a matching joint feature 328 having a plurality of negatively matching or complementary ridges and grooves prepared at a portion of the target region 304 so as to engage one another. The ridges and grooves may be oriented at an angle with respect to each other such that they are nonparallel, thereby resisting separation of the bone pieces. Alternatively, the pieces of bone may be keyed for additional interlocking.

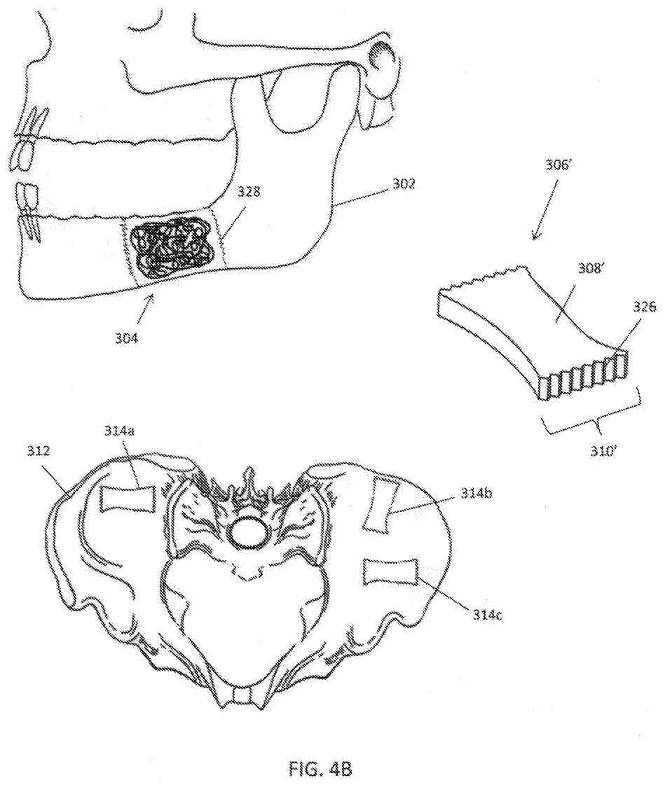

In a specific inventive embodiment, with respect to FIG. 5, a joint feature 326′ may be in the form of a dovetail joint having a tongue 330 and groove 332 pattern and a matching joint feature 328′ having an inverse matching or complementary pattern. The joint feature 326′ and matching joint feature 328′ may include any number of tongues 330 and grooves 332.

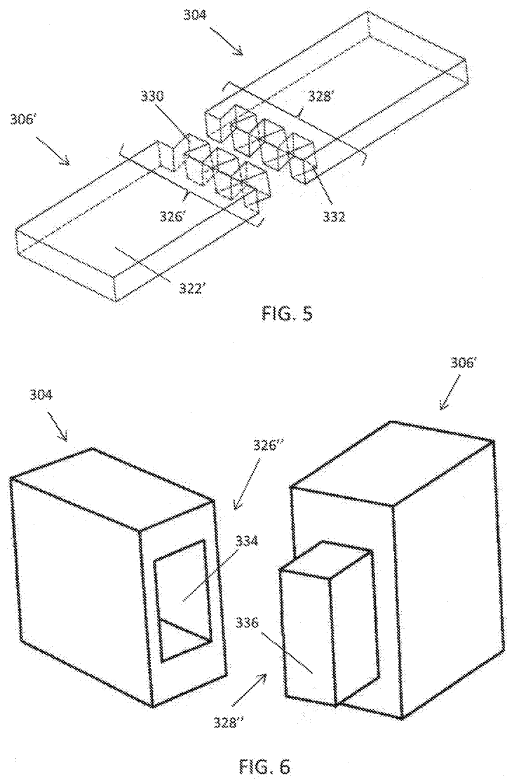

In a particular inventive embodiment, with reference to FIG. 6, a joint feature 326″ and a matching joint feature 328″ is in the form of a mortise 334 and tenon 336 where either one of the custom graft 306′ or target region 304 includes the mortise 334 or tenon 336. The tenon 336 is configured to fit in the mortise 334 to interlock the graft 306′ with the target region 304. An adhesive, such as bone cement, may be used to further secure the tenon 336 in the mortise 334. In a particular embodiment, the mortise 334 may be slightly undersized so as to wedge or press-fit the tenon 336 in the mortise 334. Although the joint shown in FIG. 6 depicts one tenon 336 and mortise 334, it should be appreciate that a plurality of tenons 336 and mortises 334 may be present to further increase stability and osseointegration. In addition, the mortise 334 may be one of a variety of shapes and forms depending on the application and user's preferences. Mortise forms operative herein illustratively include open mortise, stub mortise, through mortise, wedged half-dovetail, through-wedged half-dovetail, and combinations thereof. Likewise, the tenon 336 may also be selected from a variety of forms that illustratively include stub tenon, through tenon, loose tenon, biscuit tenon, pegged tenon, tusk tenon, teasel tenon, top tenon, and a combination thereof. The user may further design joint features having additional grooves, ridges, channels, projections or any combination thereof to form a mechanically effective and strong unit.

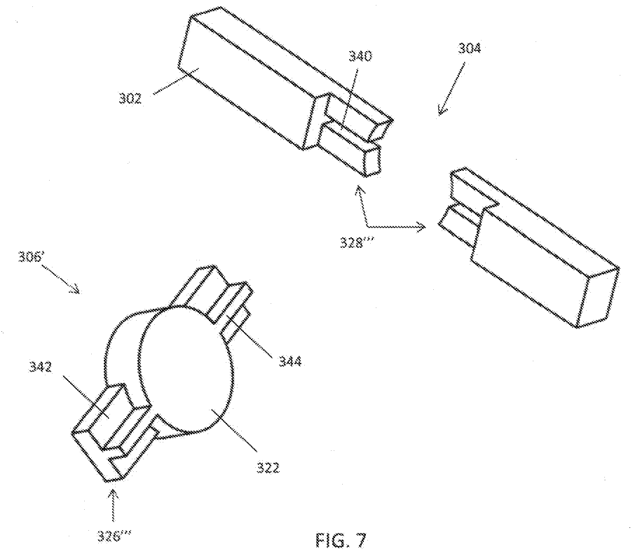

In a particular inventive embodiment, with reference to FIG. 7, a custom graft 306′ includes an inset joint feature 326″. The inset joint feature 326′″ includes an inset 342 that mates with a matching target inset feature 328″ prepared on the target region 304. The target inset 338 is preferably prepared to expose healthy bone to readily integrate with the inset 342 of the custom graft 306′. The joint feature 326′″ may further include an alignment feature 344 that interlocks with a matching alignment feature 340 prepared at the matching joint feature 328′″ of the target region 304. The alignment feature 344 allows the user to quickly identify and implant the graft 306′ in the desired planned POSE relative to the target region 304.

In another inventive embodiment, autologous bone grafting material is cored from a harvest region and subsequently packed and/or shaped to treat a target region. The user and/or planning software may identify optimal locations at the harvest region to core out the autologous bone grafting material. As described above, factors considered to identify the optimal location may include the bone quality of the grafting material, the bone quality of the surrounding bone, the reachability of the location, the surgical incision site, and the surrounding soft tissue structures. The cored bone grafting material may be crushed and directly implanted in the target region 304, or the material may be formed in a desired shape of create a custom graft (306, 306′). In a specific embodiment, the crushed grafting material may be formed in a desired shape using additive manufacture techniques with a putty or paste as an adhesive. Coring the bone grafting material at an optimal location is particularly advantageous because the incision size is minimized, and there is a reduced chance of surrounding bone morbidity.

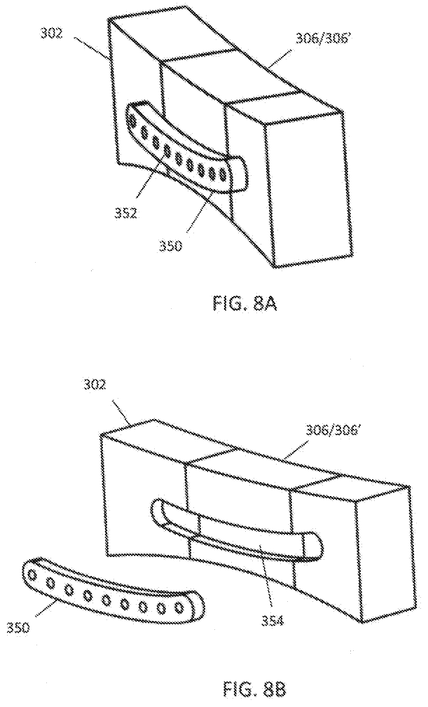

In a particular inventive embodiment, with respect to FIGS. 8A and 8B, the GUI may include tools for designing a fixation plate 350 and determining a location for the fixation plate 350 relative to the graft (306, 306′) and/or target region 304. The GUI may include off-the-shelf CAD models of typical fixation plates that the user can select and manipulate on the bone models 302. The CAD models may have modifiable surfaces to allow the user to custom design a fixation plate 350 or the user may design a custom fixation plate 350 from scratch using the same tools to design the custom graft (306, 306′) as described above. The user may further design the location and number of fixation holes 352 on the fixation plate 350 that are typically used to screw the fixation plate 350 to the graft (306, 306′) and the target region 304. In a specific embodiment, with reference to FIG. 8B, the user may define an inset 354 such that when the fixation plate 350 is inserted into the inset, the fixation plate 350 lies flush with the surrounding bone. The inset 354 is particularly advantageous because fixation plates 350 as used conventionally may protrude from the anatomy affecting the appearance of the subject. In addition, the fixation plate as used conventionally has the problem of exposure from the surrounding tissues. The custom fixation plate 350 may be ordered from an outside manufacturer or created using manufacturing techniques known in the art. In another embodiment, the fixation plate may be shaped and/or formed intra-operatively as further described during the execution step below.

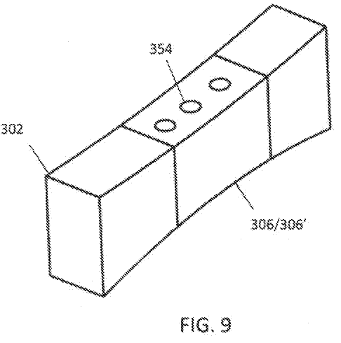

In a specific embodiment, with respect to FIG. 9, the user may design a graft (306, 306′) having object features 356. The object features 356 are designed to receive an external object. For example, if the graft (306, 306′) is for the mandible, the object features 356 may be a set of holes having internal threads and designed to receive replacement teeth, where the replacement teeth may be inserted and screwed into the holes at the same time as the grafting procedure, or during a later procedure after the graft has had time to heal.

Following the design of the custom graft (306, 306′) and the identification of an optimal harvest location 314b, a user or the planning software generates a target cut-file to prepare the target region 304 to receive the graft (306, 306′), and a harvest cut-file to harvest the graft from the identified harvesting location 314b. The target cut-file and/or harvest cut-file is executed by a computer-assisted surgical device to precisely create the graft (306, 306′) and prepare the target region. The cut-files may be optimized with regard to the dimensions and shape of the custom graft (306, 306′), the biological features surrounding the harvest location or target region, and/or the interface of the joint.

In a specific embodiment, a cut-file is generated to avoid specific tissue areas. The subject scan data may allow the user or the planning system to (a) identify the location of critical tissues, such as arteries and nerves within the intended cutting sites or the area surrounding the intended cutting site; and (b) generate cut-files that minimizes or avoids cutting or manipulation of these critical tissues. The cut-files may also have a modifiable setting where the user is able to change the parameters of the cut file intra-operatively while performing the surgery in real time to avoid the critical tissues. This is advantageous because these cut-files reduce the morbidities following the surgical procedure by avoiding the critical tissues and decreasing subject recovery time.

In a specific embodiment, a cut-file is generated with the aid of physical bone models. For complex surgical procedures, such as Dega or Salter osteotomies for pelvic correction, the physical models are used to practice, tune, and/or design mock custom grafts. The mock custom grafts may be physically manipulated by the user to determine how the graft will interact with a target region. The method is repeated until the user creates a mock graft that achieves a desired goal (e.g., structural integrity, limb alignment, or the complete replacement of a region). In a particular inventive embodiment, the user creates the mock grafts by manipulating a cutting instrument attached to a robotic arm. The robotic arm may have a mode that records the movement of the cutting instrument as the user creates the mock graft. The recorded movements are saved as a cut-file, where, intra-operatively, the robot automatically plays back the movements to create the actual custom graft. The physical bone models may be registered and tracked during the ‘practice’ surgery to ensure the robot executes the movements in the correct POSE on the subject's bone in the operating room. The physical bone models may be generated based on the scan data of the subject and additive manufacturing techniques. Several of the physical bone models may be created to allow the user plenty of opportunities to practice, tune, and/or design the mock graft.

Execution

To prepare a precise bone graft according to the plan, a computer-assisted surgical system capable of executing such precision is desirable. Examples of a computer-assisted surgical system include a 1-6 degree of freedom hand-held surgical system, an autonomous serial-chain manipulator system, a haptic serial-chain manipulator system, a parallel robotic system, or a master-slave robotic system, as described in U.S. Pat. Nos. 5,086,401, 7,206,626, 8,876,830 and 8,961,536, U.S. Pat. App. No. 2013/0060278, and PCT Intl. App. No. US2015/051713.

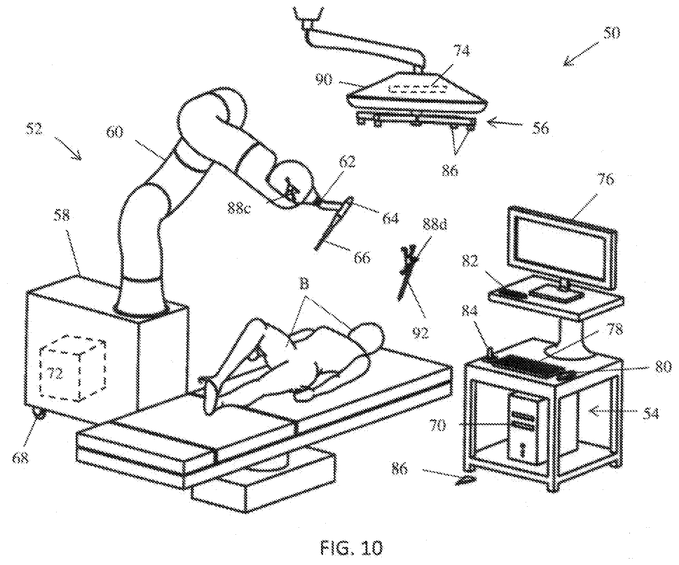

With reference to FIG. 10, a particular inventive embodiment of a robotic surgical system 50 to harvest the custom graft (306, 306′) and prepare the target region 304 is shown in the context of an operating room (OR). The surgical system 50 generally includes a surgical robot 52, a computing system 54, and an optional tracking system 56. The surgical robot 52 includes a movable base 58, a manipulator arm 60 mounted to the base 58, an end-effector flange 62 located at a distal end of the manipulator arm 60, an end-effector assembly 64 removably attached to the flange 62, and a tool 66 removably assembled to the end-effector assembly 64. The base 58 may include a set of wheels 68 to maneuver the base 58, which may be fixed into position using a braking mechanism such as a hydraulic brake. The manipulator arm 60 includes various joints and links to manipulate the tool 66 in various degrees of freedom. The joints may be prismatic, revolute, or a combination thereof. The tool 66 may be any device harvest the graft (306, 306′), prepare the target region 304, or core autologous bone coring material. For example, the tool 66 may be a burr, a saw, an end-mill, a cutter, a laser engraver, a drill, or a pin driver. The tool 66 and manipulator are controlled by commands from the computing system 54.

The computing system 54 generally includes a planning computer 70 including a processor; a device computer 72 including a processor; a tracking computer 74 including a processor, if a tracking system 56 is present; and peripheral devices. Processors operate in system 54 to perform computations associated with the inventive method. It is appreciated that processor functions are shared between computers, a remote server, a cloud computing facility, or combinations thereof. The planning computer 70, device computer 72, and tracking computer 74 may be separate entities as shown, or it is contemplated that their operations may be executed on just one or two computers depending on the configuration of the surgical system 50. For example, the tracking computer 74 may have the operational data to control the manipulator 60 and tool 66 of the surgical system 50 without the need for a device computer 72. Or, the device computer 72 may include operational data to plan the surgical procedure and design the implant without the need for the planning computer 70. In any case, the peripheral devices allow a user to interface with the surgical system components and may include: one or more user-interfaces, such as a display or monitor 76; and user-input mechanisms, such as a keyboard 78, mouse 80, pendent 82, joystick 84, foot pedal 86, or the monitor 76 may have touchscreen capabilities.

The planning computer 70 contains hardware (e.g., processors, controllers, and memory), software, data, and utilities that are dedicated to the design of the custom graft (306, 306′) and planning of a surgical procedure, either pre-operatively or intra-operatively. This may include reading medical imaging data, segmenting imaging data, constructing three-dimensional (3D) virtual models, storing computer-aided design (CAD) files, providing the GUI tools for designing the graft as described above, and generating surgical plan data (e.g., cut-files). The final surgical plan includes intra-operative operational data for modifying a volume of tissue to harvest the graft (306, 306′) and prepare the target region. The cut-file may include a set of cutting parameters such as a set of points, vectors, arm velocities, and/or arm accelerations to autonomously modify the volume of bone. The cut-file may include a set of virtual boundaries defined to haptically constrain a tool within the defined boundaries to modify the bone. The data generated from the planning computer 70 is readily transferred to the device computer 72 and/or tracking computer 74 through a wired or wirelessly connection in the operating room (OR); or transferred via a non-transient data storage medium (e.g., a compact disc (CD), a portable universal serial bus (USB) drive) if the planning computer 70 is located outside the OR.

The device computer 72 may be housed in the moveable base 58 and contain hardware, software, data and utilities that are primarily dedicated to the operation of the surgical device. This may include surgical device control, robotic manipulator control, the processing of kinematic and inverse kinematic data, the execution of registration algorithms, the execution of calibration routines, the execution of surgical plan data, coordinate transformation processing, providing workflow instructions to a user, and utilizing position and orientation (POSE) data from the tracking system 56.

The tracking system 56 of the surgical system 50 includes two or more optical receivers 86 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 88, where each fiducial marker array 88 has a unique arrangement of fiducial markers, or a unique transmitting wavelength/frequency if the markers are active LEDs. An example of an optical tracking system is described in U.S. Pat. No. 6,061,644. The tracking system 56 may be built into a surgical light 90, located on a boom, a stand, or built into the walls or ceilings of the OR. The tracking system computer 74 may include tracking hardware, software, data and utilities to determine the POSE of objects (e.g., bones B, surgical robot 52) in a local or global coordinate frame. The POSE of the objects is collectively referred to herein as POSE data, where this POSE data is readily communicated to the device computer 72 through a wired or wireless connection. Alternatively, the device computer 72 may determine the POSE data using the position of the fiducial markers detected from the optical receivers 86 directly.

The POSE data is determined using the position data detected from the optical receivers 86 and operations/processes such as image processing, image filtering, triangulation algorithms, geometric relationship processing, registration algorithms, calibration algorithms, and coordinate transformation processing. For example, the POSE of a digitizer probe 92 with an attached probe fiducial marker array 88d may be calibrated such that the probe tip is continuously known as described in U.S. Pat. No. 7,043,961. The POSE of the tool tip or tool axis of the tool 66 may be known with respect to a device fiducial marker array 88c using a calibration method as described in U.S. Prov. Pat. App. 62/128,857. The device fiducial marker 88c is depicted on the manipulator arm 60 but may also be positioned on the base 58 or the end-effector assembly 64. Registration algorithms are readily executed to determine the POSE and/or coordinate transforms between a bone B, a fiducial marker array 88, the robot 52, and a surgical plan, using the registration methods described in U.S. Pat. Nos. 6,033,415, and 8,287,522. The system 50 may further include a fluoroscopy imaging system or CT imaging system to perform image based registration as described in U.S. Pat. No. 5,951,475.

The POSE data is used by the computing system 54 during the procedure to update the coordinate transforms and/or POSEs of the bone B, the surgical robot 52, and the surgical plan to ensure the surgical robot 52 accurately executes the surgical plan on the bone B. It should be appreciated that in certain embodiments, other tracking systems may be incorporated with the surgical system 50 such as an electromagnetic field tracking system or a mechanical tracking system. An example of a mechanical tracking system is described in U.S. Pat. No. 6,308,567. In a particular embodiment, the surgical system 50 does not include a tracking system 56 and a tracked digitizer probe 92, but instead employs a mechanical digitizer arm incorporated with the surgical robot 52 as described in U.S. Pat. No. 6,033,415, and a bone fixation and monitoring system that fixes the bone directly to the surgical robot 52 and monitors bone movement as described in U.S. Pat. No. 5,086,401.

Intra-operatively, the computer-assisted system harvests the custom graft (306, 306′) and prepares the target region 304 as follows. The harvest region 312 and target region 304 are registered to the system (Blocks S224-S226 and S210-S214). The harvest region 312 may be registered first, where the system harvests the custom graft (306, 306′) prior to registering and preparing the target region 304. Conversely, the target region 304 may be registered first and prepared prior to registering the harvest region 312. Alternatively, the target region 304 and harvest region 312 are registered at the same time. The choice in registration sequence may be a function of the user's preference, and/or the reach (i.e., workspace) of the robotic arm 60. In a specific inventive embodiment, if the target region 304 is located somewhere on the skull or mandible, the registration may be accomplished using a tooth or plurality of teeth as fiducial markers, which may serve as radiopaque markers for image registration or as reference points for point-to-surface registration techniques.

With the target region 304 and/or harvest region 312 registered, the robot then either mills the custom graft (306, 306′) from the harvest region (Block S212, S228) at the identified optimal location, or prepares the target region 304 to receive the custom graft (306, 306′) (Block S216, S230). Milling is readily performed with a rotary bit engaging subject bone tissue. In a specific embodiment, the rotatory bit is less than 2 mm in diameter to create small precise shapes, and to reduce the amount of bone milled around the custom graft (306, 306′). In another embodiment, the robot harvests only a portion of the custom graft (306, 306′) so as to extract the portion of the graft from the harvesting location 314b. The extracted portion of the graft is then milled to create any additional features to complete the custom graft, ex-vivo (306, 306′).

The custom graft (306, 306′) is then implanted in the target region 304 (Block S232, S218). If the graft includes one or more joint features, the graft is implanted such that the joint features join with the matching feature on the target region 304 (Block S232) to form the joint. It is appreciated that by milling with a slight undersize the graft (306, 306′) forms a press-fit interaction with the target region 304. It is also appreciated that in the event a gap exists between the joints, or there is a need to reinforce the graft (306, 306′) to the target region 304, or a desire to improve osseointegration, the use of adhesive materials, bone fragment packing, bone growth promoters, or combinations thereof are readily available.

In another inventive embodiment, if the user designed a custom fixation plate 350 (shown in FIGS. 8A and 8B), the surgical robot 50 may shape and/or bend an off-the-shelf fixation plate to conform to the custom design. The surgical robot 50 may further mill out the specific number and location of the fixation holes 352 as designed by the user. The surgical robot 50 may further mill an inset 354 to receive the fixation plate 350. Finally, the surgical robot 50 may drill pilot holes in the graft (306, 306′) and/or the target region 304 to facilitate the insertion of screw holes or other fastening elements used to fixate the fixation plate to the graft (306, 306′) and/or target region 304.

Cadaver Bone

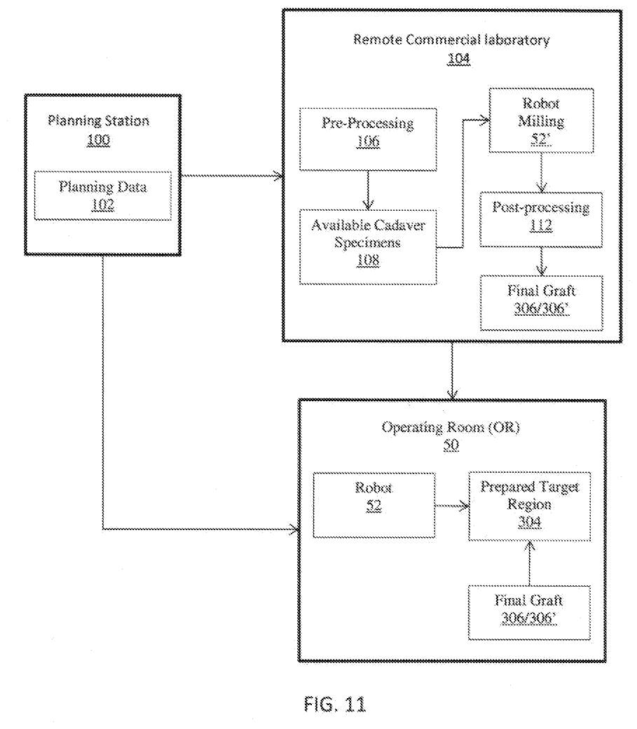

With reference to FIG. 11, in those inventive embodiments when cadaver bone is the source of the graft, planning data 102 generated at a planning station 100 having the planning software as described above is forwarded to a remote commercial laboratory 104 for preparation of the graft. The planning data 102 may include the graft dimensions and bone density requirements, the dimensions of the target region 102, and/or the entire pre-operative plan for use in the operating room 50. The remote commercial laboratory 104 may pre-process 106 the planning data 102 to verify the dimensions, review the plan, generate milling instructions, identify an optimal cadaver specimen from a database of available cadaver specimens 108, as well as on optimal location to harvest the graft from the specimen. Subsequently, the cadaver specimen is milled by a robotic system 52′. In a specific inventive embodiment, a duplicate system 52′ is used in the remote laboratory 104 for the creation of a bone graft from a cadaver harvest region relative to that used to shape a complementary target region in the operating room 50. In another embodiment, a different robot or CNC machine may be used to mill the graft. After milling, the graft is post-processed 112. The post-processing 112 may include sterilization, packaging, and optionally applying any coating or substances desired to be infiltrated into the graft, which may occur in a specialized manufacturing facility. These sets of coating or infiltrating are collectively defined herein as treating. The final graft (306, 306′) is then shipped to the situs of the surgical procedure, such as the operating room 50. The robot 52 then prepares the target region 304 to receive the remotely prepared custom graft.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes can be made in the function and arrangements of elements without departing from the scope as set forth in the appended claims and the legal equivalents thereof.

1. A method for planning a bone grafting procedure for a bone of a subject, the bone having a target region comprising:
collecting imaging scan data of the target region and of a harvest region as a source for a bone graft complementary to the target region;
generating a virtual bone model of the bone and of the harvest region;
determining with a computer processor at least one of a size, a material type, a geometry, and a position for a bone graft model complementary to the target region;
identifying a location at the harvest region for the bone graft based on the bone graft model;
registering the location of the harvest region to a first computer-assist device to harvest the bone graft;
shaping the bone graft from the harvest region with the first computer-assist device;
registering the target region to the first computer-assist device or a second computer-assist device; and
communicating cutting characteristics for the target region to the first computer-assist device or a second computer-assist device to form the target region to receive the bone graft.

2. The method of claim 1, wherein locating the identifying of the location at the harvest region for the bone graft further comprises:
superimposing the bone graft model at the harvest region in a location with good bone quality, accessible by a tool operated by the computer-assist device with minimal tissue damage, and a location with a low probability of surrounding bone morbidity after a section of bone is harvested for the bone graft.

3. The method of 1 wherein the virtual bone model is obtained by generating a three-dimensional (3-D) bone model from an image data set of the subject's bone.

4. The method of claim 3 wherein the image scan is a computed tomography (CT), dual-energy x-ray absorptiometry (DEXA), magnetic resonance imaging (MRI), X-ray scans, ultrasound, or a combination thereof.

5. The method of claim 3 wherein the scan data comprises bony structural features comprising one or more of size, shape, thickness, and curvatures.

6. The method of claim 3 wherein the scan data comprises bone property data and soft-tissue data comprising one or more of bone alignment, bone kinematics, soft tissue features, placement of nerves and arteries, bone density, and bone microarchitecture.

7. The method of claim 1 wherein a user can manipulate the virtual bone model data in a pre-operative planning software program having a graphical user interface (GUI).

8. The method of claim 7 wherein the GUI includes widgets and other tools which allow the user to manually, semi-automatically, or automatically design a custom graft, and identify a location at the harvest region to create the custom graft.

9. (canceled)

10. The method of claim 7 wherein the GUI further comprises a library of modifiable shapes resembling typical structures of a particular bone.

11. The method of claim 7 wherein the GUI is used to extract a topology of the bone model to define a set of natural contours and curvature of the subject's bone, where the contours and curvatures define at least a portion of the bone graft shape to match the actual bone topology of the subject.

12. (canceled)

13. The method of claim 7 wherein the GUI further comprises boundary tools to define boundaries of the bone graft model.

14. (canceled)

15. (canceled)

16. The method of claim 7 wherein the GUI further comprises an indicator to indicate a percentage of overlap between an outline of the bone graft model and a set of potential harvesting locations.

17. The method of claim 1 further comprising providing features to the bone graft and the target region to at least one of improve the connectivity of the graft with the target region or to aid in aligning the bone graft with the target region.

18. (canceled)

19. (canceled)

20. The method of claim 1 wherein the bone graft is autologous.

21. The method of claim 1 further comprising harvesting the bone graft from the identified location and to the specifications of the graft model geometry using said processor or another processor.

22. The method of claim 1 further comprising treating said bone graft prior to mating to the target region.

23. The method of claim 1 wherein the harvest region in the bone with the target region.

24. The method of claim 1 wherein the harvest region is in the same type of bone as the bone containing the target region and is in cadaver bone.

25. The method of claim 1 further comprising implanting the bone graft into the target region.

26. A surgical system for performing the computerized method of claim 1, the system comprising:
a surgical robot;
at least one workstation, each of the at least one workstations comprising a computer, user-peripherals, and a monitor for displaying the graphical user interface (GUI);
wherein the computer further comprises the processor, non-transient storage memory, and other hardware, software, data and utilities to harvest a bone graft complementary to a target region; and
wherein the user peripherals allow a user to interact with the GUI and include user input mechanisms comprising at least one of a keyboard, mouse, or a touchscreen capability on the monitor.
